(19) United States
(12) Patent Application Publication
(10) Pub. No.: US 2015/0152351 A1
(43) Pub. Date: Jun. 4, 2015

(54) PERFUME COMPOSITION FOR REPRODUCING FRAGRANCE OF ABIES KOREANA OF JEJU ISLAND

(71) Applicant: AMOREPACIFIC CORPORATION, Seoul (KR)

(72) Inventors: Ji Young CHOI, Yongin-si (KR); Seung Ho KO, Yongin-si (KR); Chang Sik WOO, Yongin-si (KR)

(21) Appl. No.: 14/555,833

(22) Filed: Nov. 28, 2014

(30) Foreign Application Priority Data
Nov. 29, 2013 (KR) 10-2013-0147611
Nov. 24, 2014 (KR) 10-2014-0164552

(51) Int. Cl. C11B 9/00; A61K 8/34; A61Q 17/04; A61K 8/31

(52) U.S. Cl. CPC C11B 9/0034; C11B 9/0015; C11B 9/0007; A61K 8/31; A61K 8/34; A61Q 17/04

(57) ABSTRACT

Provided is a perfume composition having excellent acceptability and also reproducing the distinct fragrance of Abies Koreana of Jeju Island by adding trans-4,8-dimethyl-1,3,7-nonatriene to limonene, linalool, and myrcene, that are analyzed as the fragrance ingredients of Abies Koreana of Jeju Island by a SPME method.

### TECHNICAL FIELD

The present invention relates to a perfume composition that reproduces the distinct fragrance of Abies Koreana of Jeju Island, and has excellent acceptability.

### BACKGROUND

Abies Koreana is a special product that belongs to Abies firma, Pinaceae and grows only in Korea, and is distributed over high mountains that are located at an altitude of 500 m to 2000 m in the southern province, such as, Halla Mountain, Deogyu Mountain, and Jiri Mountain. Abies Koreana is an evergreen needle-like leaf tree and stands 10 to 15 m. It has beautiful type of trees, and thus, may be planted as decorative tree. The needle-like leaf thereof is short, and has split ends and re-entrants, and the back side of the leaf has two air hole lines. It is monoecism, and in June, 5 to 10 light brown oval blossoms having 1 cm in length come out as a male flower, and a strong purple and a little longer blossom than the male flower comes out as a female flower. In September, greenish brown or purplish brown cylinder pine cone fruits grow to be straight for the sky. Because of its beautiful type of trees and little size, it may be planted for being used as a Christmas tree, and thus, it is known as a “Korean Fir”. Currently, Abies Koreana is either extinct or threatened with extinction, so that it was designated a red list of endangered species in International Union for Conservation of Nature (IUCN). Abies Koreana is quite rare as the species that are typical landscape factor and also tree species informing climate change of Halla Mountain, and are distributed over a limited area as endemic species.

As a method for exhibiting the fragrance of Abies Koreana, there may be many methods. For example, as a general method, there are a solvent extraction method including preparing a fragrance ingredient of Abies Koreana in an absolute type with a solvent and a steam distillation method including making a fragrance ingredient of Abies Koreana into an essential oil. However, these methods need to be applied with high heat during an extraction process, and it is a complex process to do and too much time is spent on the processes. Therefore, the fragrance thus obtained may be largely different with the original fragrance of Abies Koreana.

Recently, in order to supplement these disadvantages, a head space method and a solid phase micro extraction (SPME) method were developed. Among them, the SPME method was disclosed in the thesis (SUPELCO international, Vol. 13, No. 4, p. 9 to 10), which is incorporated in the present specification as a reference. According to the SPME method, unlike the conventional extraction methods, the fragrance ingredients can be quickly and simply analyzed without using a solvent and performing a pre-treating process. However, when the main ingredients of the fragrances thus analyzed are only used for preparing a fragrance, similarity is low and acceptability is also insufficient, and thus, there may be a limit for exhibiting the fragrance of an object.

### CITATION LIST

### Patent Document

Patent Document 1: Korean Patent Publication No. 2008-0096995 (Publication Date: Nov. 4, 2008)

### SUMMARY

Accordingly, the present inventors analyzed the main ingredients of the fragrance of Abies Koreana using a SPME method that is useful for analyzing a fragrance substance of a living natural substance. However, as a result of performing a sensory test, the present inventors found that the fragrance of Abies Koreana cannot be reproduced only using the main ingredients of the fragrance analyzed.

In addition, the present inventors found that when the main ingredients of the fragrance are included, and also trans-4,8-dimethyl-1,3,7-nonatriene (E-4,8-dimethyl-1,3,7-nonatriene) is used therefor, the fragrance of Abies Koreana can be reproduced, and excellent acceptability can be exhibited. Therefore, the present inventors completed the present invention.

Therefore, an object of the present invention is to provide a perfume composition capable of reproducing the distinct fragrance of Abies Koreana and exhibiting excellent acceptability.

Another object of the present invention is to provide an external application skin composition including the perfume composition.

In order to achieve the above objects, the present invention provides a perfume composition having the reproduced fragrance of Abies Koreana by including the fragrance ingredients of Abies Koreana and trans-4,8-dimethyl-1,3,7-nonatriene as an active component.

In addition, the present invention provides an external application skin composition including the perfume composition having the reproduced fragrance of Abies Koreana.

### DETAILED DESCRIPTION

The present invention provides a perfume composition having the reproduced fragrance of Abies Koreana, which can reproduce the fragrance of Abies Koreana and can exhibit excellent acceptability by including the fragrance ingredients of Abies Koreana and trans-4,8-dimethyl-1,3,7-nonatriene as an essential component.

Hereinafter, the present invention will be described in more detail.

The composition according to the present invention includes, as the fragrance ingredients of Abies Koreana, the main ingredients and secondary ingredients, in which the main ingredient includes limonene, linalool, and myrcene. As the secondary ingredients, bornyl acetate, 4-mentha-1(7),8-dien-2-one, trans-4-mentha-2,8-dien-1-ol, and alpha pinene are used.

In addition, in order to increase the similarity of the fragrance of Abies Koreana and exhibit excellent acceptability of the fragrance of Abies Koreana by reproducing the distinct fragrance of Abies Koreana, the perfume composition of the present invention uses, as an essential ingredient, the fragrance ingredients of Abies Koreana described above, and trans-4,8-dimethyl-1,3,7-nonatriene as an artificial synthetic material.

The perfume composition of the present invention may include 48.00 to 68.00 Wt % of limonene, 0.10 to 20.00 wt % of linalool, 0.10 to 20.00 wt % of myrcene, and 0.01 to 15.00 wt % of trans-4,8-dimethyl-1,3,7-nonatriene that are the above-described main ingredients with respect to the total weight of the composition. In addition, the total content of the main ingredients may be 50 to 90 wt % and preferably 60 to 80 wt % with respect to the total weight of the composition.

The perfume composition of the present invention includes, as the secondary ingredients, the amounts of 0.01 to 16.00 wt % of bornyl acetate, 0.01 to 14.00 wt % of 4-mentha-1(7),8-dien-2-one, 0.01 to 14.00 wt % of trans-4-mentha-2,8-dien-1-ol, and 0.01 to 13.00 wt % of alpha pinene with respect to the total weight of the composition.

When the limonene, linalool, myrcene, and trans-4,8-dimethyl-1,3,7-nonatriene are used in the range that are out of the above-described range as the main ingredients for reproducing the fragrance of Abies Koreana, the similarity of the fragrance of Abies Koreana decreases and the acceptability of the fragrance of Abies Koreana decreases, and thereby, it is not preferable. However, it is preferable to use bornyl acetate, 4-mentha-1(7), 8-dien-2-one, trans-4-mentha-2,8-dien-1-ol, and alpha pinene, which were the secondary ingredients, in the above-described range, but it does not greatly affect the fragrance of Abies Koreana, and thus, the amounts being out of the above-described range may be included as long as it does not affect the reproduction of the fragrance of Abies Koreana.

According to the present invention, the composition prepared by including the ingredients in the above-described range may be mixed in an external application skin formulation, such as, a perfume and cosmetics, and the combined amounts may be properly selected and combined in order to achieve the desired effects according to the general technology in the related art.

The examples of these external application formulations may include, but is not limited thereto, ointment, lotion, solubilizer, suspension, emulsion, creams, gels, spray, cataplasma, medicated pads, patches, or pain relief medication, but any kinds of the formulations that are known in the related art may be combined.

Meanwhile, as a method for analyzing the fragrance ingredients of Abies Koreana, a SPME method may be used in the present invention. According to the SPME method, the fragrance ingredients adsorbed into a fiber can be quickly subjected to desorption and injection into the GS-MS column without a specific pre-treating process, and thus, the time for analyzing can be significantly shortened. Therefore, the SPME method is useful for analyzing the fragrance ingredients of Abies Koreana.

In the present invention, a mixed perfume is composed based on the fragrance ingredients of Abies Koreana analyzed by the SPME method, and then, for the mixed perfume, the similarity and acceptability of the fragrance of Abies Koreana are confirmed by performing an olfactory sensory test. The sensory test is performed by the specialist perfumers and ordinary people, and the similarity and acceptability of the fragrance of Abies Koreana are estimated through carrying out a survey.

Hereinafter, the present invention will be described in more detail with reference to Examples, but the present invention is not limited thereto. The other applications and modifications of the present invention can be understood by a person who skilled in the related art.

### REFERENCE EXAMPLE 1

### Analysis of Fragrance of Abies Koreana Using SPME Method

A fresh tree of Abies Koreana having a strong scent was selected, and then, the fragrance ingredients were collected through the absorption of the fragrance ingredients into a fiber coated with polyacrylate (polyacrylate fiber) in 85 μm for 2 hours. The collection was performed for 3 hours at Seogwipo-si, Jeju Island.

After collecting the fragrance ingredients (Sample A), the fiber was sealed, transferred and put into an inlet of a GC-MS, was subjected to desorption for 2 minutes, and then, the GC-MS analysis was performed. The analyzing conditions by GC-MS are as follows.

Analyzing Conditions
Analyzing Instrument: HP 5890 II GC
Detector: HP 5972 MSD
Column: DB-1 (60 m×0.25 mm×0.25 μm) (manufactured by Agilent)
Carrier Gas: He
Injection Temperature: 250° C.
Detector Temperature: 280° C.
Oven Temperature: 70° C. to 220° C. (3° C./min.)
Ionization Voltage: 70 eV
Desorption Time: 2 min.

As a result, the fragrance ingredients of Abies Koreana thus analyzed by the SPME method are listed in the following Table 1.

TABLE 1

| Name of fragrances | Content (wt %) |
|---|---|
| Limonene | 59.86 |
| linalool | 7.48 |
| myrcene | 3.78 |
| Bornyl acetate | 2.84 |
| 4-mentha-1(7),8-dien-2-one | 1.98 |
| trans-4-mentha-2,8-dien-1-ol | 1.94 |
| Alpha pinene | 1.85 |
| Others | 20.27 |

As shown in the above Table 1, it can be confirmed that Abies Koreana has limonene, linalool, and myrcene as a main fragrance ingredient.

### TEST EXAMPLE 1

### Evaluation of Comparison Sensory Test Between Mixed Perfumes of the Fragrance Ingredients Analyzed and Fragrances of Abies Koreana

Based on the above-analyzed result, the new mixed perfume (Sample B) having the following compositions listed in Table 2 was prepared, and then, the similarity of the fragrances between the above fragrance and natural Abies Koreana were confirmed through a sensory test.

TABLE 2

| Name of fragrances | Content (wt %) |
|---|---|
| Limonene | 59.86 |
| linalool | 7.48 |
| myrcene | 3.78 |
| Bornyl acetate | 2.84 |
| 4-mentha-1(7),8-dien-2-one | 1.98 |
| trans-4-mentha-2,8-dien-1-ol | 1.94 |
| Alpha pinene | 1.85 |
| DPG | 20.27 |
| Total content | 100 |

The sensory test was performed by using 20 general men and women in an age of 20 to 45 as an object. The above general men and women smelled each of the scents of Sample A having the fragrance ingredients that were directly collected from a flowerpot of Abies Koreana and Sample B having the mixed perfume reproduced by a SPME method in Reference Example 1, and then, recorded their answers on the questionnaire as the following Table 3, thereby investigating the similarity (Question 1) and acceptability (Question 2) of the fragrances of Sample A and Sample B. The results thus obtained are listed in the following Table 4.

TABLE 3
Questionnaire

(1) Question 1: How similar the fragrances of Sample A and Sample B
are?
1) Completely different each other.
2) Less likely to be similar.
3) Just so-so.
4) A few similarities.
5) Very similar each other.
(2) Question 2: How much do you like the fragrance of Sample B?
1) Didn't like it
2) Not very good
3) just so-so
4) A little better
5) Really good

TABLE 4

| | Question 1 | Question 2 |
|---|---|---|
| Object | (Similarity) | (Acceptability) |
| 1 | 1 | 3 |
| 2 | 1 | 2 |
| 3 | 2 | 1 |
| 4 | 2 | 2 |
| 5 | 2 | 2 |
| 6 | 2 | 2 |
| 7 | 2 | 1 |
| 8 | 4 | 1 |
| 9 | 2 | 2 |
| 10 | 2 | 2 |
| 11 | 2 | 2 |
| 12 | 3 | 2 |
| 13 | 2 | 3 |
| 14 | 1 | 3 |
| 15 | 3 | 1 |
| 16 | 2 | 1 |
| 17 | 2 | 3 |
| 18 | 1 | 2 |
| 19 | 2 | 3 |
| 20 | 2 | 1 |
| Mean | 2.00 | 1.95 |

As shown in the above Table 4, it can be confirmed that there are many differences between Sample B, the mixed perfume, and Abies Koreana planted and Sample B exhibits low acceptability.

### TEST EXAMPLE 2

### Fragrance Analysis by Specialist Evaluators and Comparison with Fragrance of Abies Koreana

As seen in the above Test Example 1, it can be confirmed that the perfume composition prepared by using the ingredients analyzed by the SPME method does not have the similarity of the fragrance as compared with the fragrance of Abies Koreana. Therefore, the specialist evaluators composed of perfumers performed the sensory test about each of the fragrance ingredients of Abies Koreana. As a result, it can be confirmed that among the fragrance ingredients of Abies Koreana, three ingredients, such as, limonene producing a fresh lemon fragrance, linalool producing a herbal floral fragrance, and myrcene producing a sweet balsam fragrance are main ingredients producing the distinct fragrance of Abies Koreana.

### EXAMPLE 1

### Preparation of New Mixed Perfume According to Content Change of Main Ingredients

According to Test Example 2, it can be confirmed that the main ingredients of the original fragrance of Abies Koreana are limonene, linalool, and myrcene. Therefore, the present inventors made #1 to #4 new mixed perfumes in order to prepare the fragrances having good acceptability and also exhibiting the fragrance of Abies Koreana as it is by including these three ingredients.

The #1 to #4 mixed perfumes were prepared by constantly maintaining the rate of these three ingredients and changing the contents of three fragrances in the range of 65 to 80 wt %. The detailed compositions are listed in the following Table 5.

TABLE 5

| Mixed perfume No. | | | |
|---|---|---|---|
| #1 | #2 | #3 | #4 |

| | | | | |
|---|---|---|---|---|
| Name of perfume (Content: wt %) | 65% | 70% | 75% | 80% |
| Limonene | 54.71 | 58.92 | 63.12 | 67.34 |
| linalool | 6.84 | 7.36 | 7.89 | 8.41 |
| myrcene | 3.45 | 3.72 | 3.99 | 4.25 |
| Bornyl acetate | 11.54 | 9.89 | 8.25 | 6.59 |
| 4-menthan-1(7),8-dien-2-one | 8.05 | 6.9 | 5.75 | 4.6 |
| trans-4-mentha-2,8-dien-1-ol | 7.89 | 6.76 | 5.63 | 4.51 |
| Alpha pinene | 7.52 | 6.45 | 5.37 | 4.3 |
| Total content | 100.0 | 100.0 | 100.0 | 100.0 |

### TEST EXAMPLE 3

### Evaluation of Sensory Test of Fragrances Prepared in Example 1

For new mixed four perfumes of Example 1, the sensory tests for confirming the similarity and acceptability as compared with the fragrance of Abies Koreana were performed using the same method as Example 1. Meanwhile, after performing the comparison sensory test for two mixed perfumes, the break time was maintained for 5 minutes so as not to generate after-sensation paralysis. The results of sensory test for confirming similarity and acceptability of the new mixed perfume and the fragrance of Abies Koreana are listed in the following Table 6.

TABLE 6

| | Question 1 (Similarity) | | | | Question 2 (Acceptability) | | | |
|---|---|---|---|---|---|---|---|---|
| | Mixed perfume No. | | | | Mixed perfume No. | | | |
| Object | #1 | #2 | #3 | #4 | #1 | #2 | #3 | #4 |
| 1 | 2.0 | 4.0 | 3.0 | 2.0 | 1.0 | 3.0 | 3.0 | 2.0 |
| 2 | 3.0 | 3.0 | 3.0 | 2.0 | 2.0 | 3.0 | 3.0 | 2.0 |
| 3 | 3.0 | 3.0 | 4.0 | 3.0 | 2.0 | 4.0 | 3.0 | 1.0 |
| 4 | 2.0 | 3.0 | 3.0 | 2.0 | 3.0 | 3.0 | 2.0 | 2.0 |
| 5 | 2.0 | 4.0 | 2.0 | 2.0 | 4.0 | 3.0 | 3.0 | 1.0 |
| 6 | 2.0 | 4.0 | 3.0 | 2.0 | 3.0 | 4.0 | 2.0 | 2.0 |
| 7 | 2.0 | 4.0 | 2.0 | 3.0 | 3.0 | 2.0 | 1.0 | 2.0 |
| 8 | 2.0 | 3.0 | 3.0 | 2.0 | 3.0 | 3.0 | 2.0 | 1.0 |
| 9 | 2.0 | 4.0 | 3.0 | 3.0 | 3.0 | 3.0 | 3.0 | 2.0 |
| 10 | 3.0 | 4.0 | 2.0 | 2.0 | 2.0 | 4.0 | 3.0 | 1.0 |
| 11 | 2.0 | 3.0 | 4.0 | 2.0 | 3.0 | 3.0 | 2.0 | 2.0 |
| 12 | 3.0 | 4.0 | 3.0 | 3.0 | 2.0 | 3.0 | 3.0 | 3.0 |
| 13 | 3.0 | 2.0 | 2.0 | 2.0 | 2.0 | 3.0 | 2.0 | 2.0 |
| 14 | 3.0 | 2.0 | 3.0 | 3.0 | 2.0 | 2.0 | 3.0 | 3.0 |
| 15 | 2.0 | 4.0 | 3.0 | 3.0 | 2.0 | 3.0 | 3.0 | 3.0 |
| 16 | 2.0 | 3.0 | 3.0 | 2.0 | 2.0 | 3.0 | 3.0 | 2.0 |
| 17 | 2.0 | 3.0 | 3.0 | 2.0 | 3.0 | 3.0 | 3.0 | 3.0 |
| 18 | 3.0 | 4.0 | 3.0 | 1.0 | 2.0 | 4.0 | 3.0 | 3.0 |
| 19 | 3.0 | 4.0 | 3.0 | 2.0 | 3.0 | 4.0 | 3.0 | 2.0 |
| 20 | 2.0 | 3.0 | 3.0 | 2.0 | 2.0 | 3.0 | 2.0 | 3.0 |
| Mean | 2.40 | 3.40 | 2.90 | 2.25 | 2.45 | 3.15 | 2.60 | 2.10 |

As shown in the above Table 6, it can be confirmed that Sample #2 having the total content of the three main ingredients of 70 wt % shows the most outstanding similarity and acceptability among four mixed perfumes, but both the similarity and acceptability fall short of their expectations.

### EXAMPLE 2

### Preparation of Fragrance Having Improved Fragrance by Analysis of Special Evaluation Group

In the above Table 6, in order to improve similarity and acceptability of Sample #2 showing the fragrance that was most similar to the fragrance of Abies Koreana, like the above-described Sample #2, new mixed perfume was prepared by adding limonene, linalool, and myrcene in the content of 70 wt %, and then, adding trans-4,8-dimethyl-1,3,7-nonatriene giving an unique fragrance reproduced exhibiting the fragrance of Abies Koreana thereto. At this time, the content standard of trans-4,8-dimethyl-1,3,7-nonatriene was determined by the decision of perfumer specialist group, and thus, trans-4,8-dimethyl-1,3,7-nonatriene was added in the various contents, such as, 0.01 wt %, 1.00 wt %, 5.00 wt %, 10.00 wt %, 15.00 wt %, and 20.00 wt %.

TABLE 7

| Name of fragrance (content: wt %) | #A | #B | #C | #D | #E | #F |
|---|---|---|---|---|---|---|
| Limonene | 58.92 | 58.92 | 58.92 | 58.92 | 58.92 | 58.92 |
| Linalool | 7.36 | 7.36 | 7.36 | 7.36 | 7.36 | 7.36 |
| Myrcene | 3.72 | 3.72 | 3.72 | 3.72 | 3.72 | 3.72 |
| Trans-4,8-dimethyl-1,3,7-nonatriene | 0.01 | 1.00 | 5.00 | 10.00 | 15.00 | 20.00 |
| Bornyl acetate | 9.88 | 9.56 | 8.28 | 6.59 | 4.94 | 3.30 |
| 4-mentha-1(7),8-dien-2-one | 6.90 | 6.67 | 5.61 | 4.60 | 3.45 | 2.30 |
| Trans-4-mentha-2,8-dien-1-ol | 6.76 | 6.53 | 5.67 | 4.51 | 3.38 | 2.25 |
| Alpha pinene | 6.45 | 6.24 | 5.44 | 4.3 | 3.23 | 2.15 |
| Total content | 100.0 | 100.0 | 100.0 | 100.0 | 100.0 | 100.0 |

### TEST EXAMPLE 4

### Sensory Test of Mixed Perfumes Prepared in Example 2

For evaluating acceptabilities of the fragrances and similarities of mixed perfumes, such as, #A, #B, #C, #D, #E, and #F mixed in Example 2 with respect to Abies Koreana, the sensory test was performed using the same method as Test Example 1. After performing the comparison sensory test for two mixed perfumes, the break time was maintained for 5 minutes so as not to generate after-sensation paralysis. The results of sensory test for confirming similarity and acceptability of the new mixed perfumes and the fragrance of Abies Koreana are listed in the following Table 8.

TABLE 8

| | Question 1 (Similarity) | | | | | | | Question 2 (Acceptability) | | | | | | |
|---|---|---|---|---|---|---|---|---|---|---|---|---|---|---|
| | Mixed perfume No. | | | | | | | Mixed perfume No. | | | | | | |
| | No | | | | | | | No | | | | | | |
| Object | addition | #A | #B | #C | #D | #E | #F | addition | #A | #B | #C | #D | #E | #F |
| 1 | 4.0 | 4.0 | 4.0 | 3.0 | 4.0 | 4.0 | 3.0 | 3.0 | 4.0 | 3.0 | 3.0 | 3.0 | 4.0 | 4.0 |
| 2 | 3.0 | 4.0 | 4.0 | 4.0 | 4.0 | 3.0 | 3.0 | 3.0 | 3.0 | 3.0 | 4.0 | 4.0 | 4.0 | 3.0 |
| 3 | 3.0 | 3.0 | 5.0 | 4.0 | 5.0 | 4.0 | 3.0 | 4.0 | 4.0 | 4.0 | 5.0 | 4.0 | 5.0 | 3.0 |
| 4 | 3.0 | 3.0 | 4.0 | 4.0 | 4.0 | 4.0 | 3.0 | 3.0 | 4.0 | 4.0 | 4.0 | 4.0 | 4.0 | 2.0 |
| 5 | 4.0 | 3.0 | 4.0 | 3.0 | 4.0 | 3.0 | 3.0 | 3.0 | 3.0 | 4.0 | 5.0 | 3.0 | 4.0 | 3.0 |
| 6 | 4.0 | 3.0 | 4.0 | 4.0 | 3.0 | 3.0 | 3.0 | 4.0 | 3.0 | 3.0 | 4.0 | 4.0 | 4.0 | 4.0 |
| 7 | 4.0 | 4.0 | 3.0 | 3.0 | 4.0 | 4.0 | 3.0 | 2.0 | 4.0 | 3.0 | 4.0 | 3.0 | 3.0 | 3.0 |
| 8 | 3.0 | 4.0 | 3.0 | 4.0 | 4.0 | 4.0 | 4.0 | 3.0 | 4.0 | 4.0 | 3.0 | 4.0 | 3.0 | 4.0 |
| 9 | 4.0 | 3.0 | 4.0 | 3.0 | 4.0 | 4.0 | 3.0 | 3.0 | 4.0 | 3.0 | 5.0 | 3.0 | 4.0 | 3.0 |
| 10 | 4.0 | 4.0 | 4.0 | 4.0 | 5.0 | 3.0 | 4.0 | 4.0 | 3.0 | 4.0 | 4.0 | 4.0 | 4.0 | 3.0 |
| 11 | 3.0 | 3.0 | 4.0 | 5.0 | 3.0 | 3.0 | 3.0 | 3.0 | 3.0 | 3.0 | 4.0 | 5.0 | 4.0 | 4.0 |
| 12 | 4.0 | 4.0 | 3.0 | 4.0 | 5.0 | 4.0 | 4.0 | 3.0 | 4.0 | 4.0 | 4.0 | 4.0 | 3.0 | 3.0 |
| 13 | 2.0 | 4.0 | 2.0 | 4.0 | 3.0 | 3.0 | 3.0 | 3.0 | 3.0 | 3.0 | 4.0 | 4.0 | 2.0 | 2.0 |
| 14 | 2.0 | 3.0 | 3.0 | 3.0 | 5.0 | 3.0 | 3.0 | 2.0 | 3.0 | 5.0 | 3.0 | 3.0 | 3.0 | 2.0 |
| 15 | 4.0 | 4.0 | 4.0 | 4.0 | 4.0 | 4.0 | 4.0 | 3.0 | 4.0 | 4.0 | 4.0 | 4.0 | 4.0 | 3.0 |
| 16 | 3.0 | 4.0 | 4.0 | 4.0 | 4.0 | 4.0 | 3.0 | 3.0 | 4.0 | 3.0 | 5.0 | 4.0 | 4.0 | 3.0 |
| 17 | 3.0 | 3.0 | 4.0 | 4.0 | 4.0 | 3.0 | 3.0 | 3.0 | 3.0 | 5.0 | 4.0 | 4.0 | 4.0 | 4.0 |
| 18 | 4.0 | 4.0 | 3.0 | 4.0 | 4.0 | 4.0 | 4.0 | 4.0 | 4.0 | 4.0 | 4.0 | 4.0 | 3.0 | 2.0 |
| 19 | 4.0 | 4.0 | 4.0 | 4.0 | 4.0 | 4.0 | 4.0 | 4.0 | 4.0 | 4.0 | 5.0 | 4.0 | 4.0 | 3.0 |
| 20 | 3.0 | 3.0 | 4.0 | 4.0 | 4.0 | 4.0 | 3.0 | 3.0 | 4.0 | 3.0 | 5.0 | 4.0 | 4.0 | 4.0 |
| Mean | 3.40 | 3.55 | 3.70 | 3.80 | 4.05 | 3.60 | 3.30 | 3.15 | 3.60 | 3.65 | 4.15 | 3.80 | 3.70 | 3.10 |

As seen in Table 8, it can be confirmed that when 0.01 to 20.00 wt % of trans-4,8-dimethyl-1,3,7-nonatriene is added to the main fragrance ingredients of Abies Koreana, which are composed of limonene, linalool, and myrcene, the fragrance of Abies Koreana can be reproduced, and acceptability can be high (mean 3.0 or more). Especially, it can be confirmed that when 0.01 to 15.00 wt % of trans-4,8-dimethyl-1,3,7-nonatriene is included, the acceptability of the fragrance and similarity of the fragrance to Abies Koreana are most outstanding (mean 3.5 or more).

Meanwhile, it can be confirmed that when of trans-4,8-dimethyl-1,3,7-nonatriene is not included or is included in the amount of higher than 20.00 wt %, the similarity and acceptability of the fragrance are decreased.

### EXAMPLE 3

### Preparation of Mixed Perfume

The mixed perfumes, such as, #G, #H, #I, and #J, were prepared according to the compositions listed in the following Table 9. The mixed perfumes, such as, #G, #H, #I, and #J, included limonene, linalool, and myrcene that were the main ingredients in the same contents as #B, #C, #D, and #E in Example 2, but included the contents of bornyl acetate, 4-mentha-1(7),8-dien-2-one, trans-4-mentha-2,8-dien-1-ol, and alpha pinene, secondary ingredients, that were differently changed.

TABLE 9

| | Mixed perfume No. | | | |
|---|---|---|---|---|
| Name of perfume (content: wt %) | #G | #H | #I | #J |
| Limonene | 58.92 | 58.92 | 58.92 | 58.92 |
| Linalool | 7.36 | 7.36 | 7.36 | 7.36 |
| Myrcene | 3.72 | 3.72 | 3.72 | 3.72 |
| Trans-4,8-dimethyl-1,3,7- | 1.00 | 1.00 | 1.00 | 1.00 |
| nonatriene | | | | |
| Bornyl acetate | 25.00 | 2.00 | 1.00 | 1.00 |
| 4-mentha-1(7),8-dien-2-one | 2.00 | 25.00 | 2.00 | 1.00 |
| Trans-4-mentha-2,8-dien-1-ol | 1.00 | 1.00 | 25.00 | 2.00 |
| Alpha pinene | 1.00 | 1.00 | 1.00 | 25.00 |
| Total content | 100.0 | 100.0 | 100.0 | 100.0 |

### TEST EXAMPLE 5

### Sensory Test of Mixed Perfumes Prepared in Example 3

For evaluating acceptabilities of the fragrances and similarities of mixed perfumes, such as, #G, #H, #I, and #J, mixed in Example 3 with respect to Abies Koreana, the sensory test was performed using the same method as Test Example 1. After performing the comparison sensory test for two mixed perfumes, the break time was maintained for 5 minutes so as not to generate after-sensation paralysis. The results of sensory test for confirming similarity and acceptability of the new mixed perfumes and the fragrance of Abies Koreana are listed in the following Table 10.

TABLE 10

| | Question 1 (Similarity) | | | | Question 2 (Acceptability) | | | |
|---|---|---|---|---|---|---|---|---|
| | Mixed perfume No. | | | | Mixed perfume No. | | | |
| Object | #G | #H | #I | #J | #G | #H | #I | #J |
| 1 | 4.0 | 4.0 | 4.0 | 3.0 | 3.0 | 4.0 | 4.0 | 4.0 |
| 2 | 4.0 | 3.0 | 4.0 | 4.0 | 3.0 | 3.0 | 4.0 | 5.0 |
| 3 | 5.0 | 4.0 | 5.0 | 4.0 | 4.0 | 4.0 | 5.0 | 4.0 |
| 4 | 4.0 | 4.0 | 4.0 | 4.0 | 4.0 | 3.0 | 4.0 | 4.0 |
| 5 | 4.0 | 3.0 | 4.0 | 3.0 | 4.0 | 4.0 | 4.0 | 4.0 |
| 6 | 4.0 | 3.0 | 4.0 | 4.0 | 3.0 | 4.0 | 3.0 | 3.0 |
| 7 | 3.0 | 4.0 | 3.0 | 3.0 | 3.0 | 3.0 | 4.0 | 4.0 |
| 8 | 3.0 | 4.0 | 3.0 | 4.0 | 4.0 | 4.0 | 4.0 | 4.0 |
| 9 | 4.0 | 4.0 | 4.0 | 3.0 | 3.0 | 3.0 | 4.0 | 4.0 |
| 10 | 4.0 | 3.0 | 4.0 | 4.0 | 4.0 | 4.0 | 5.0 | 5.0 |
| 11 | 4.0 | 3.0 | 4.0 | 5.0 | 3.0 | 5.0 | 3.0 | 3.0 |
| 12 | 3.0 | 4.0 | 3.0 | 4.0 | 4.0 | 4.0 | 5.0 | 5.0 |
| 13 | 2.0 | 3.0 | 2.0 | 4.0 | 3.0 | 4.0 | 3.0 | 3.0 |
| 14 | 3.0 | 3.0 | 3.0 | 3.0 | 5.0 | 3.0 | 5.0 | 5.0 |
| 15 | 4.0 | 4.0 | 4.0 | 4.0 | 4.0 | 4.0 | 4.0 | 4.0 |
| 16 | 4.0 | 4.0 | 4.0 | 4.0 | 3.0 | 4.0 | 4.0 | 4.0 |
| 17 | 4.0 | 3.0 | 4.0 | 4.0 | 5.0 | 4.0 | 4.0 | 4.0 |
| 18 | 3.0 | 4.0 | 3.0 | 4.0 | 4.0 | 4.0 | 4.0 | 4.0 |
| 19 | 4.0 | 4.0 | 4.0 | 4.0 | 4.0 | 4.0 | 4.0 | 4.0 |
| 20 | 4.0 | 4.0 | 4.0 | 4.0 | 3.0 | 4.0 | 4.0 | 4.0 |
| Mean | 3.70 | 3.60 | 3.70 | 3.80 | 3.65 | 3.80 | 4.05 | 4.05 |

As seen in Table 10, it can be confirmed that even though the contents of bornyl acetate, 4-mentha-1(7),8-dien-2-one, trans-4-mentha-2,8-dien-1-ol, and alpha pinene, which were the secondary ingredients,are changed, in the case of being the contents of limonene, linalool, myrcene, and trans-4,8-dimethyl-1,3,7-nonatriene, which are the main ingredients, in the range according to the present invention, the similarity and acceptability are excellent without major influence.

### PREPARATION EXAMPLE 1

The perfume shown in the following Table 11 was prepared by combining the fragrance composition having the similarity for the fragrance of Abies Koreana.

TABLE 11
Perfume including perfume composition
having reproduced fragrance of Abies Koreana
(content: wt %)

| | |
|---|---|
| Mixed perfume having reproduced fragrance of Abies | 15 |
| Koreana | |
| Alcohol | TO 100 |
| Water | 2 |
| Pigment | Suitable amount |
| Sunscreen agent | Suitable amount |

As fully discussed hereinbefore, the perfume composition according to the present invention can reproduce the fragrance of Abies Koreana and can also improve acceptability of the fragrance by including limonene, linalool, and myrcene that are the main ingredients of the fragrance of Abies Koreana and trans-4,8-dimethyl-1,3,7-nonatriene in order to reproduce the fragrance of Abies Koreana analyzed by a SPME method.

While this invention has been particularly shown and described with reference to an exemplary embodiment thereof, it will be understood by those skilled in the art that various changes in form and details may be made therein without departing from the spirit and scope of this invention as defined by the appended claims.

1. A perfume composition having reproduced fragrance of Abies Koreana by including main fragrance ingredients of Abies Koreana of Jeju Island, and trans-4,8-dimethyl-1,3,7-nonatriene, as an active ingredient.

2. The perfume composition of claim 1, wherein the main ingredients of Abies Koreana of Jeju Island are composed of limonene, linalool, and myrcene.

3. The perfume composition of claim 2, wherein the composition further includes one or more selected from the group consisting of bornyl acetate, 4-mentha-1(7),8-dien-2-one, trans-4-mentha-2,8-dien-1-ol, and alpha pinene.

4. The perfume composition of claim 2, wherein the perfume composition includes in the amounts of 48.00 to 68.00 wt % of limonene, 0.10 to 20.00 wt % of linalool, 0.10 to 20.00 wt % of myrcene, and 0.01 to 15.00 wt % of trans-4,8-dimethyl-1,3,7-nonatriene, with respect to the total weight of the composition.

5. The perfume composition of claim 2, wherein the perfume composition includes the amounts of 50 to 90 wt % of the main ingredients, and 0.01 to 15.00 wt % of trans-4,8-dimethyl-1,3,7-nonatriene, with respect to the total weight of the composition.

6. The perfume composition of claim 3, wherein the perfume composition includes the amount of 0.01 to 16.00 wt % of bornyl acetate with respect to the total weight of the composition.

7. The perfume composition of claim 3, wherein the perfume composition includes the amount of 0.01 to 14.00 wt % of 4-mentha-1(7),8-dien-2-one with respect to the total weight of the composition.

8. The perfume composition of claim 3, wherein the perfume composition includes the amount of 0.01 to 14.00 wt % of trans-4-mentha-2,8-dien-1-ol with respect to the total weight of the composition.

9. The perfume composition of claim 3, wherein the perfume composition includes the amount of 0.01 to 13.00 wt % of alpha pinene with respect to the total weight of the composition.

10. An external application skin composition comprising the perfume composition according to claim 1.
